(19) United States
(12) Patent Application Publication
(10) Pub. No.: US 2021/0345974 A1
(43) Pub. Date: Nov. 11, 2021

(54) METHOD AND SYSTEM OF CAREGIVER ALERT FOR PATIENT BREATHING STATUS DURING SLEEP IN A HOME ENVIRONMENT TO PREVENT COVID-19 RELATED DEATH

(71) Applicant: Mi Kyung Chung, Durham, NC (US)

(72) Inventor: Mi Kyung Chung, Durham, NC (US)

(21) Appl. No.: 15/929,563

(22) Filed: May 11, 2020

(51) Int. Cl. A61B 5/00; A61B 5/1455; A61B 5/024

(52) U.S. Cl. CPC A61B 5/746; A61B 5/0004; A61B 5/0022; A61B 5/14552; A61B 5/6826; A61B 5/747; A61B 5/02405; A61B 5/4818; A61B 5/7455; A61B 5/7282; A61B 5/681

(57) ABSTRACT

Many people have died in their homes in their sleep while suffering from COVID-19 infection. This invention proposes a patient and caregiver bracelet system by which patient breathing and heart rate status declines are monitored through pulse oximetry connected to their bracelets and caregivers sleeping in the same house are alerted via their own caregiver bracelets. The underlying communications signaling can be sent via wireless digital technology and also via radio frequency.

### BACKGROUND OF THE INVENTION

Many people have died in their homes in their sleep while suffering from COVID-19 infection, and some of these people were in the prime of their lives, ranging from ages 30 to 45 years of age. Some of these people had their significant others and other family members sleeping in different rooms of the same house, or sometimes in the same room. These patients' family members had no way of knowing that the patients' breathing status was declining in the middle of the night during sleep, because there was no alert system designed, implemented or activated to notify them or other healthcare system entities that said patient breathing status was declining.

This method and system of alert, as presented in this nonprovisional patent application, may prevent a COVID-19 related death that may have been preventable, if only family members or other system partners had been alerted in a timely fashion as to the breathing status of the patient, in the middle of the night when the patient and family members are all sleeping. The author of this nonprovisional patent application spent many nights in a hospital cardiac unit as a nurse watching over sleeping patients' heart and breathing rates. Also, she spent many nights on a children's hospital unit watching over sleeping babies and children's heart and breathing rates. When the nurse is alerted to the decline in patient breathing status, the nurse role is to inspect such patients and often times wake them up, perform further assessment and call for additional help.

Also, within the hospital environment, these patients were often connected to monitors watched over by external off-nursing floor central watch stations, manned by live human being personnel who would also serve as “back-up watchers” of heart and breathing rates, in the event that none of the nurses or doctors on the floor could attend to the alert event when it suddenly occurred. These off-nursing floor external central watch stations often were composed of groups of people sitting in a room filled with monitors, simply watching for heart and breathing anomalies, all night long. In the event that any of the monitors demonstrated any heart rate or breathing rate anomalies, the central watch station employees would call the relevant nurse's station to understand what was happening with the patient and also to take additional actions as defined by preestablished hospital protocols. These preestablished protocols are often refined and updated based on clinical effectiveness measures, real-world outcome studies and all manner of other kinds of scientific studies.

The author of this patent application also watched over her four children every time they became sick with respiratory disease, all night long without sleeping herself, because of a mother's worry about her children's breathing during sleep when they were ill with respiratory conditions. In the event that such a portable, mobile, fully functional alert system becomes widely available, patients and caregivers may be able to sleep with less anxiety overall during pandemic related respiratory illnesses. Such systems may prevent COVID-19 related or other future pandemic related deaths of patients who died while sleeping in their own homes.

### BRIEF SUMMARY OF THE INVENTION IN 5 PARTS

Introduction: This nonprovisional patent application via omnibus specification (since no one compartmentalized specification makes any sense as a “standalone invention”) is being submitted only a few days after the USPTO announced the COVID-19 patent accelerated review process, in the hopes that manufacturers, healthcare systems, computer technology companies, government regulators, health insurance companies and many other stakeholders, can work together to quickly develop such an alert system or a similar alert system to allow family members to be alerted to their loved ones' decline in breathing status during sleep. There are 5 distinct parts of this nonprovisional utility patent application and therefore 5 claims. The following is a brief overview of each of the 5 Parts of this patent application.

Part 1: For the patient, a fingernail-based pulse oximeter is attached to a bracelet that the patient wears. This portable pulse-oximeter bracelet device will contain a digital wireless component to be able to deliver signals to other connected devices such as their personal mobile phone and the caregiver's alert reception bracelets.

Part 2: The caregiver will also wear a bracelet that is connected digitally to the patient's bracelet, and when the patient's heart rate or pulse oximetry oxygen reading dips below the thresholds as established by prescribing physicians when the device is prescribed, the caregiver will receive an alert vibration and noise from their own bracelet to notify them of a potential anomaly in the patient's breathing.

Part 3: In addition to the caregiver alert, the digital alert will also be delivered to the Central Breathing Status Watch Station, which will be a center established with human beings who are on call 24 hours a day, 365 days a week, to watch breathing status measurements and heart rate measurements for each bracelet that is prescribed. In the event that the breathing or heart rate status presents an anomaly, not only will family members be alerted through their bracelet alerts, but this Central Breathing Status Watch Station will also receive an alert to take measures in notifying first responders as appropriate.

Part 4: Due to the imperfections of digital wireless remote access technology, especially for those who live in rural areas or are otherwise lacking the necessary resources for Internet connection, the patient and caregiver alert bracelet system will also contain a robust alert mechanism using two-way radio transmission technology, that can serve as a back-up in the potential failure of the digital wireless system and also can serve as an initial alert system for those who lack connection to high speed Internet.

Part 5: Lastly, a probable reason why such a system has not already been invented and implemented widely for home use for patients, caregivers and central watch stations is perhaps because of the potential patient death liability issue. Even if such a system were designed, launched and implemented in wide home use to mitigate for pandemic COVID-19 related deaths at home during sleep, manufacturers and service providers may be very wary of the liability implications of actually working together to develop solutions. Therefore, there is a very sad possibility that this institutional fear of liability prevents very large well-capitalized companies all across the planet from developing such systems and launching them to market. To properly structure corporate and organizational incentives for the benefit of our nation's public health, the author of this patent also recommends that governmental authorities, including officials from the FDA, who must approve such product and system for use as “Digital Health Software” (“Software as a Medical Device” aka “SaMD”), take additional legislative initiatives regarding managing product and device liability in collaboration with manufacturers, health service providers, information technology product and service providers as well as with health insurance companies. The legislative precedent that could be followed is that of vaccine related product liability, for which the US government assumes a special form of liability funding responsibility through the National Childhood Vaccine Injury Act.

### DETAILED DESCRIPTION OF THE INVENTION IN 5 PARTS

PART 1 DETAILED DESCRIPTION: This patent application involves a method and system by which a fingernail-based pulse oximeter is attached to a bracelet that the patient wears and this portable pulse-oximeter bracelet device will contain a digital wireless component to be able to deliver signals to other connected devices such as their personal mobile phone and the caregiver's alert reception bracelets, based on triggers defined by thresholds of expected normal breathing status parameters and normal heart rate parameters, as defined by the patient's physician (FIG. 1: System Components). The alert signal going to the patient or caregiver mobile phone is meant also to be delivered via mobile wireless carrier signals or via internet cable modem/fiber optic cable technology to the remote “central watch station.” As part of the software, medically acceptable algorithms must be coded and included to determine acceptable thresholds of heart rates and breathing rates, as designated by the prescribing physician, in terms of what data events would serve as potential triggers to pushout system alerts that are then delivered to caregivers via an alert/alarm system. As to the threshold values that determine trigger data events, for example, there are extremely athletic patients whose “normal” heart rates may actually lie in the “bradycardic” range for what “normal patients” of that same age group, gender and weight group would normally exhibit. For those individuals, physicians may select to choose a different “threshold of alarm” since those patients' “norm” in terms of basal heart rate is actually much lower than the usual patient. Based on patient clinical variability, such flexibility must be designed into this system to allow for such physician directed threshold set ups. Since this patent application refers to a method and system involving the use of clinical data in conjunction with the proper set up of information technology algorithms, the FDA is likely to demand to see some sort of “real world” evidence studies to further ensure the efficacy and effectiveness of such solutions. Although, at this point, after so many people in the prime of their years have died of COVID-19 related deaths in their sleep, we have perhaps arrived at the point in our nations' history that we collectively accept that “something is better than nothing.” In other words, family members of loved ones who are sick potentially with COVID-19 (since not everyone who died in their sleep even got tested for COVID-19 despite presenting with the appropriate symptomology at their respective healthcare institutions), may wish to know in one way or another that their loved ones' breathing has declined in the middle of the night during sleep, whether it is from an alarm noise or vibration from an alarm bracelet, an alarm or vibration from their smart phone or an actual call from a central watch station official.

PART 2 DETAILED DESCRIPTION: The caregiver will also wear a bracelet that is connected digitally or via radio frequency to the patient's pulse oximetry and heart rate bracelet, and when the patient's heart rate or pulse oximetry oxygen reading and breathing rate dips below the thresholds as established by prescribing physicians when the device is prescribed, the caregiver will receive an alert vibration and noise from their own bracelet to notify them of a potential anomaly in the patient's breathing (FIG. 2: Trigger, Signal, Data Transmission Method). Upon receipt of such alert, the caregiver can call first responders and also attempt to inspect the patient and provide aid as appropriate. Those who are the wearers of the caregiver bracelet must also be provided the necessary HIPPA and other privacy training, as well as agree to other terms of conditions as to how such devices should be used for the fiduciary duty of caring for the patient. There is also a very large liability component for the caregivers in question, since many caregivers of elderly or persons with disabilities may be employed by professional home health companies or other professional group home entities or other similar organizations that provide aid for patients in their homes. This liability issue must be also addressed via legislation, as will be further discussed in claim 5.

PART 3 DETAILED DESCRIPTION: In addition to the caregiver alert, the digital alert will also be delivered to the Central Breathing Status Watch Station, which will be a center established with human beings who are on call 24 hours a day, 365 days a week, to watch breathing status measurements and heart rate measurements for each bracelet that is prescribed. In the event that the breathing or heart rate status presents an anomaly, not only will family members be alerted through their bracelet alerts, but this Central Breathing Status Watch Station will also receive an alert to take measures in notifying first responders as appropriate. This claim involves the actual physical development of a real life “Central Breathing Status Watch Station” that is manned by live, trained human beings at all times over the 24-hours, 365 days, during the entire year. Such “Central Breathing Status Watch Stations” may have to be manned by people who are licensed for various roles, depending on state laws, since some state laws may indicate that those technicians who are responsible for watching telemetry monitoring equipment must be licensed and trained in a particular way. Regardless, these “Central Breathing Status Watch Stations” should be housed in centralized facilities with their human being employees showing up to work at the actual physical facility in question, without the possibility of “remote work-from-home” since the entire point of the “Watch Station” is to make sure there are human beings who are truly awake in the middle of the night to serve as back up watchers for family members of sick patients. For those employees of the “Central Breathing Status Watch” stations, during their “Non-Watch Shift” days, they can be free to “work from home” and perform other work duties, such as training, development, etc. Similar to “911 Operators” these employees must be trained and prepared to handle the life and death implications of how they perform their duties during their active “Watch Shifts.”

PART 4 DETAILED DESCRIPTION: Due to the imperfections of digital wireless remote access technology, especially for those who live in rural areas or are otherwise lacking the necessary resources for Internet connection, the patient and caregiver alert bracelet system will also contain a robust alert mechanism using two-way radio transmission technology, that can serve as a back-up in the potential failure of the digital wireless system and also can serve as an initial alert system for those who lack connection to high speed Internet (FIG. 3: System Components Based on Radio Transmission Based Alert System). In addition, there are many people (patients and caregivers) who may voluntarily refuse to “connect” to a “Central Watch Station” either due to privacy issues or personal preference, and they must also be provided a way to be alerted in the event of a decline in the patient's breathing status. Access to such monitoring system must NOT be made conditional to patient and caregivers' agreement to be forcibly connected to any “Central Watch Station,” because it may be the case that the patients and family members choose to perform that monitoring themselves and alert first responders themselves in the event of a breathing or heart rate anomaly. In the case of a digital wireless connection failure, or in the case of voluntary refusal to be connected in a digitally wireless manner to any central watch station, by embedding two-way radio frequency technology into the patient and caregiver alert bracelets can enable a non-digital wireless backup mechanism to fulfill the same caregiver alert and alarm functionality based on patient decline in breathing status and heart rate changes (FIG. 4: Method of Data Transmission via Radio Transmission Alert System). A challenge in implementing this solution in real life, is that of fitting in two-way radio technology into a light weight portable wearable technology, and also the challenge of mitigating for radio frequency wave signal emission and reception in the interfering presence of microwaves and internet wireless device waves that are being deployed in the same vicinity and even within the same patient and caregiver bracelets. A potential mitigating mechanism may be to completely shut off the digital signal deployment mechanism once the radio transmission is activated. However, to reiterate the patent author's earlier point that “something is better than nothing,” many families of patients possibly ill with COVID-19 may prefer to wear a bulky heavy bracelet around during their sleep, if such a device could alert them to their loved one's breathing status in the middle of the night. Also, this two-way radio frequency technology is even of greater utility and importance for people who cannot afford regular high-speed internet connectivity services, as well as for people who live in very rural areas with limited or spotty high-speed internet or mobile carrier coverage. Also, by including this two-way radio frequency backup technology into the patient and caregiver bracelets, the internet connective service providers themselves may assume less pressure regarding patient outcome liability. Otherwise, in the event of poor patient outcomes in the use of such method and system, patients and families may assign liability to the Internet Service Provider whose internet connections may have been slowed down due to some completely unrelated problem. For example, in the event of a major construction project in the patient's city, in which construction work digging involved the unintentional and accidental severing of several fiber optic cable lines, some patient's internet connection could suddenly become disconnected. If the only signal and alert system is based solely on digital wireless technology, then such internet service providers must assume the entirety of liability risk. However, if the two-way radio frequency signaling and alert system is built into the bracelets, the internet service providers themselves may sense less of such liability pressure and may perhaps be more willing to collaborate with all the other stakeholder entities to develop a workable solution.

PART 5 DETAILED DESCRIPTION: Lastly, a probable reason why such a home-based caregiver alert system has not already been invented and implemented widely for home use for patients, caregivers and remote backup central watch stations is perhaps because of the potential patient death liability involved. Even if such a system were designed, launched and implemented in wide home use to mitigate for pandemic COVID-19 related deaths at home during sleep, manufacturers and service providers may be wary of the liability implications of actually working together to develop solutions. Even in the case of 1 patient death liability suit, stakeholder entities could quickly become bankrupt and potentially have to forfeit their continuing existence and an ongoing business operation. Therefore, there is a very sad possibility that this institutional and organizational fear of liability prevents very large and very well-capitalized companies all across the planet from developing such home-based alert systems and launching them to market. Therefore, to properly structure corporate incentives to innovate for the benefit of our nation's public health, and to mitigate for the possibility of more loved ones dying of COVID-19 related illness during sleep while caregivers are also sleeping under the same roof, this author of this patent also recommends that governmental authorities, including officials from the FDA, who must approve such product and system for use as “Digital Health Software” (“Software as a Medical Device” aka “SaMD”), take additional legislative initiatives regarding managing product and device liability in collaboration with manufacturers, health service providers, information technology product and service providers as well as with health insurance companies. The legislative precedent that could be followed is that of vaccine related product liability, for which the US government assumes a special form of liability funding responsibility through the National Childhood Vaccine Injury Act. Perhaps the related legislation could be titled the “National Home Breathing Monitoring System Injury Act” or some similar nomenclature. Such an Act would prevent the full liability tort related suits from bankrupting healthcare service companies, health information technology vendors, medical device manufacturers, pulse-oximetry component manufacturers, home health caregiver companies, group home caregiver entities, mobile communications carriers, radio frequency technology service providers, and a long list of other stakeholders involved to make such a caregiver alert system a reality for our country. If any of the key stakeholders are afraid that their respective organizational entities would not financially survive the potential product or service liability tort actions, due to patient deaths (that are occurring now anyway, without any such home caregiver alert systems whatsoever) then those stakeholders, despite being large, powerful and well-capitalized, may actively seek to avoid developing requisite solutions or otherwise come up with politically palatable reasons why developing such solutions is not feasible.

### BRIEF DESCRIPTION OF DRAWINGS (FIGS. 1-4)

FIG. 1: System Components for Digital Wireless Internet Based Patient and Caregiver Alarm Bracelet System. This drawing represents the primary components of the digital patient fingernail pulse oximetry meter attached to patient bracelet system, based on a digital wireless internet service provider hosted cloud data storage and data processing system, connecting the patient, patient mobile phone, caregiver, caregiver mobile phone, and the remote monitoring watch center and its employees. FIG. 1 also highlights the real world scenario of patients ill with COVID-19 attempting to recuperate in their homes from the illness, and sleeping often times in separate rooms from their significant others in order to allow the healthy significant other to sleep more peacefully without having to listen to the noise related to the sick patient's potential coughing fits. While such separation during sleep is recommended from a public health standpoint to prevent potential infection of the healthy partner, such social distancing during sleep also prevents monitoring of the patient's active health condition and breathing status by the healthy partner. To mitigate this caregiver monitoring gap, as caregivers sleep in physically distanced quarters for the sake of their own health, this invention has been outlined in detail in this patent application.

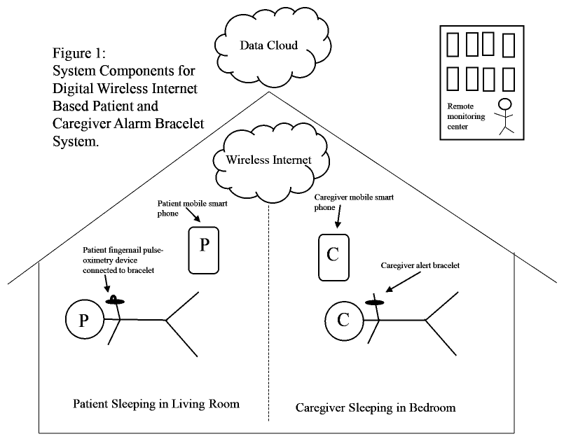

FIG. 2: Method and Process of Establishing Triggers, Signals, and Data Transmission (Follow the Arrows). This drawing shows the actual method by which the movement of trigger signals and data to and from various components of the system being described are deployed. Each step of data or signal movement is numbered with the corresponding descriptions labeled accordingly.

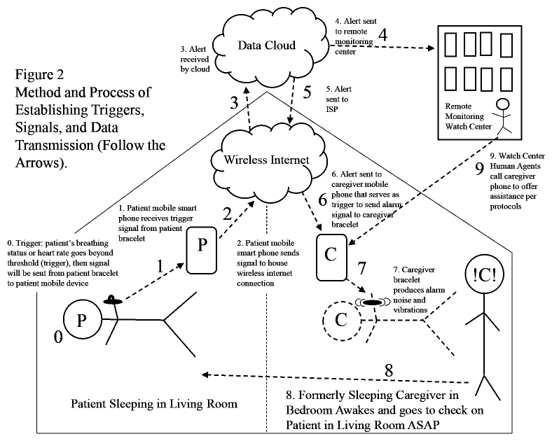

FIG. 3: System Components Based on Radio Transmission Alert System. In real life, in the absence of digital wireless internet connection, how would such a caregiver alarm or alert bracelet system work? Also, in the event of an internet connect failure, which could happen due to weather related events (hurricane, tornadoes, snowstorms, floods, lightning, hail, etc.), how would the caregiver alert system still work? In addition, in the event that there are patients and caregivers who choose to exercise their right to opt out of their patient data being exposed to the digital internet connection and remote watch center monitoring systems, how else could their caregivers still be alerted or alarmed in the event that patient's breathing or heart rate status were to decline during sleep?

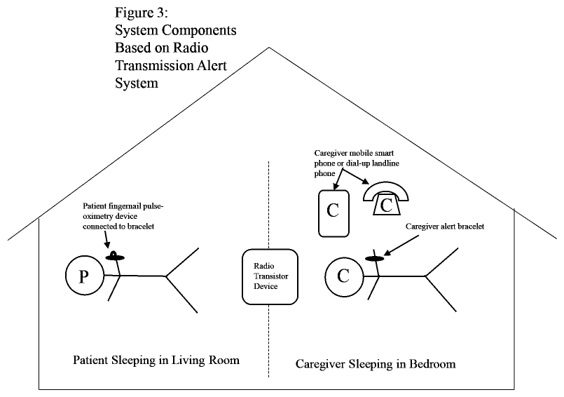

FIG. 4: Method and Process of Data Transmission via Radio Transmission Alert System. This drawing describes the various steps of how and in what order certain triggers will be activated and then what signals will be sent where in order for the caregiver to be alerted about the patient's decline in breathing status or heart rate status, without being connected to the digital wireless technology offered by internet service providers. Also, this radio frequency based signaling and alert system can also be used as a backup mechanism in the event that the digital internal wireless technology is for some reason experiencing an interruption or is not functioning fast enough.

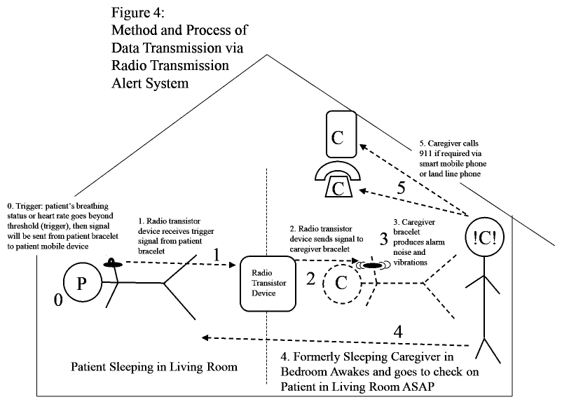

1: This patent application claims to invent a method and system by which a fingernail-based pulse oximeter is attached to a bracelet that the patient wears and this portable pulse-oximeter bracelet device will contain a digital wireless component to be able to deliver signals to other connected devices such as their personal mobile phone and the caregiver's alert reception bracelets, based on triggers defined by thresholds of expected normal breathing status parameters and normal heart rate parameters, as defined by the patient's physician.

2: This patent application claims to invent a method and system by which the caregiver will also wear a bracelet that is connected digitally or via radio frequency to the patient's pulse oximetry and heart rate bracelet, and when the patient's heart rate or pulse oximetry oxygen reading and breathing rate dips below the thresholds as established by prescribing physicians when the device is prescribed, the caregiver will receive an alert vibration and noise from their own bracelet to notify them of a potential anomaly in the patient's breathing

3: This patent application claims to invent a method and system by which a digital alert regarding the decline of patient's breathing status and heart status will be delivered to the Central Breathing Status Watch Station, which will be a center established with human beings who are on call 24 hours a day, 365 days a week, to watch breathing status measurements and heart rate measurements for each patient bracelet that is prescribed and in the event that the breathing or heart rate status presents an anomaly, not only will family members be alerted through their bracelet alerts, but this Central Breathing Status Watch Station will also receive an alert to take measures in notifying first responders and communicating with caregivers as appropriate.

4: This patent application claims to invent a method and system by which the patient and caregiver alert bracelet system will contain a robust alert mechanism using two-way radio transmission technology, that can serve as a back-up in the potential failure of the digital wireless system and also can serve as an initial alert system for those who lack connection to high speed Internet or those who refuse to connect to the wireless interact system due to privacy or personal preference rationale.

5: This patent application claims to invent a method and system by which multiple stakeholders necessary participation, to develop this solution to provide caregivers a way to be alerted in the middle of the night or any other time of day during which the patient and caregiver is asleep, is encouraged and incentivized by instituting rationale legislation to protect such entities to the full extent of the financial consequences of liability in the event of tort actions being brought forward by future parties claiming injury.
